(19) United States
(12) Patent Application Publication
(10) Pub. No.: US 2016/0008155 A1
(43) Pub. Date: Jan. 14, 2016

(54) Noninvasive Method and Apparatus for Facilitating Weight Loss

(71) Applicant: Simon Ourian, Beverly Hills, CA (US)

(72) Inventor: Simon Ourian, Beverly Hills, CA (US)

(21) Appl. No.: 14/330,998

(22) Filed: Jul. 14, 2014

(51) Int. Cl. A61F 5/00

(52) U.S. Cl. CPC A61F 5/0009

(57) ABSTRACT

In accordance with one embodiment, an apparatus and method for facilitating weight loss are provided which constitute improvements over prior surgical treatments by providing a proven way to promote desirable weight loss in overweight patients through induced appetite reduction. The invention is comfortable enough to be used everyday without causing undue agitation to the wearer and provides measurable reductions in appetite and increased weight loss.

### CROSS-REFERENCE TO RELATED APPLICATIONS

None.

### NOTICE OF COPYRIGHT AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### TECHNICAL FIELD

The present invention relates to methods for stimulating weight loss and, in particular, to using a compression apparatus which, when worn, creates a compression to the wearers skin, underlying tissues/muscles, and ultimately the stomach. The methods and apparatus described herein have been proven to encourage weight loss when used in conjunction with a health care professional-prescribed healthy diet.

### DISCUSSION OF THE RELATED ART

The incidence of excessive weight gain potentially leading to obesity is on the rise. What has been described as an epidemic by some health experts is a leading cause of preventable death in the United States currently ranked as high as cigarette smoking. In order to address this pervasive issue, a variety of treatment options have been proposed by those in the art.

The most traditional approaches have comprised nutritional counseling combined with exercise and education. However, the majority of these have demonstrated relatively little long term success. This has led many to turn to more aggressive, invasive, and sometimes irreversible approaches to weight loss. These invasive surgeries include various stomach banding or gastroplasty ring devices employed for gastric restriction to physically reduce the size of the stomach thereby directly reducing the amount of food a patient can take in.

A number of these methods have been patented. For example, U.S. Pat. No. 4,592,339 (Mentor Corporation), U.S. Pat. Nos. 5,074,868, 5,226,429 and 5,601,604 (Inamed Development Co.) and U.S. Pat. Nos. 5,771,903 and 6,102,922 (Kirk Promotions Limited). Some of the known gastric bands have incorporated an inflatable member for adjusting the diameter of the stoma opening created by the band. Surgery to provide either gastric restriction or malabsorption in cases of severe obesity have shown the greatest success long-term, but are major surgical procedures that can lead to both physiological and emotional side effects. As a result, these treatments are often reserved for more extreme cases while other avenues have been explored by those struggling with traditional weight loss methods.

Garments which apply force to a particular part of the body are commonly used in medical compression therapy. Compression garments have been used post-operatively following various surgeries with results shown to dramatically reduce edema, shorten recovery, skin shrinkage, and achieve the positive reduction results much earlier than had no compression device been employed. In these embodiments, the compression garment is used to facilitate drainage and prevent accumulation of fluid within the subcutaneous tissue. These garments have proven effective at restoring the body to its original condition when worn for a number of weeks after a surgery.

Compression therapy has been used in a variety of different ways to facilitates the bodies own natural processes. For example, in U.S. Pat. No. 5,005,567, a loosely-knit sock is first pulled over the wound dressing material and then an elastic sock with a low coefficient of friction is pulled over the first understocking between stockings, with the application of the second stocking being facilitated due to the low friction between the stockings.

Advances in the technology behind these garments has encouraged there use in the medical arena. Many of the heretofore known compression garments consist of a fiber count fabric composition that provides support and elasticity. For instance, a number of compression garments are constructed from 80% nylon and 20% elastic memory fabric, such as that available under the trademark SPANDEX. The weave of the garment fabric facilitates dissipation of body heat and transfer of moisture away from the body.

Other compression garments have been proposed to provide enhanced directional stability are typically designed to shape the body. For instance, U.S. Pat. No. 3,752,164 issued to the Playtex Corporation discloses a compression garment providing multiple-region abdominal control. This garment forms a girdle with vertically distensible spaced apart lines of “zig-zag stitching” which helps resist horizontal stresses. U.S. Pat. No. 3,621,849 discloses a therapeutic garment for maternity use which is intended to be adapted to be worn by pregnant women for providing shapely abdominal support. This garment is directed to the pregnancy stage and does not provide for post-pregnancy support.

The prior art fails to disclose a means by which these garments may be utilized to facilitate weight loss in people. Currently, there exists a need for a method and apparatus that will combine presently available advances in these garments with medical expertise to provide optimal weight loss results while avoiding the invasive surgical alternatives. The present invention effectuates these needs.

### SUMMARY

An apparatus and method for facilitating weight loss are provided which constitute improvements over prior surgical treatments by providing a proven way to promote desirable weight loss in overweight patients through induced appetite reduction. Methods of the invention generally include applying a desired amount of topical pressure to the region of the body defined by the mid-sternum to the pubic bone, hereafter referred to as the mid-abdomen.

Pressure may be applied via a mechanical compression apparatus, respectively, encircling the mid-abdomen. By way of example and not limitation, such a compression apparatus may be an elastic compression garment capable of being worn underneath a persons clothing. Maximum epidermal or topical compression is applied to the wearer's stomach through forces originating at the compression apparatus.

The anatomical region receiving the compression apparatus is comprised of areas which, when pressure is applied, mimic the feeling of being satiated making the wearer feel full and reducing their desire to ingest more food. The compression apparatus is designed to cover only the essential parts of the body in a manner proven to facilitate weight loss. Thus, ensuring it can be worn for a prolonged period of time such as during the entire workday.

The present invention allows for a specially designed compression garment that, typically, when worn for approximately twelve hours per day, will result in an identifiable reduction in appetite by the wearer and increased weight loss when compared with individuals attempting to loss weight without the aid of the compression apparatus of the present invention.

The compression apparatus is designed to keep the skin, tissues, and muscles firmly pressed against the mid-abdomen. In mothers recovering from weight gains associated with pregnancy, the reduction of the loose skin, tissues, and muscle mass facilitated by the the compression apparatus have been shown to lead to a faster return for the individual to their pre-pregnancy figure.

In one embodiment of the present invention, the compression apparatus may be a garment with a body encircling region comprised of an elastic two-way stretch material such as that available under the trademark SPANDEX. The garment is worn so as to cause directional compression to the mid-abdomen portion of the wearers body resulting in a comfortable level of compression permitting it to be worn throughout the day without any substantial agitation to the wearer. The only difference the wearer should feel is a noticeable reduction in appetite resulting from the targeted pressure of the garment.

The size of the garment is adjustable to the size of the wearer. In one embodiment, the garment may comprise a vertical opening, such as with a girdle, to facilitate wearing and removing the garment. The opening may be closed using a variety of different means including, but not limited to, zippers, buttons, hook-and-loop fasteners, string, and the like.

Moreover, adjustments can be made to the amount of pressure applied by the garment to the wearers body based on the individuals tolerances. A medical practitioner may also make any necessary adjustments to enhance or reduce the amount of force applied by the garment in order to facilitate the needs of their patient. As the stomach rescinds, the compression may be adjusted to the adequate level for achieving optimized results.

The compression element may comprise a variety of different means for securing the garment on the wearer's body and adjusting the amount and manner in which pressure is applied. By way of example, and not limitation, the garment may comprise a plurality of adjustment members. In one embodiment, these members may comprise adjustment straps located on each side of the garment. In another embodiment, the adjustment straps may be replaced with zippers. In yet another embodiment of the present invention, hook-and-loop fasteners may be used to facilitate adjustment of the compression apparatus. The adjustment members facilitate various degrees of compression allowing the wearer or medical practitioner to compensate for the reduction of in size by simply moving the adjustment member to the desired degree of compression.

In a preferred embodiment of the present invention, pressure is applied circumferentially rather than vertically. Put another way, the pressure characteristics of the garment are oriented in a horizontal direction with respect to the trunk of the wearer. This ensures that, when the compression garment is worn around the mid-abdomen, pressure travels from proximal epidermal and muscular tissue to the more distal target area of the stomach. Thus, mimicking the sensation of being full without the wearer eating as much as they are typically accustomed to.

Furthermore, it is envisioned that, in another embodiment, a single compression material that is not enhanced in any particular area may be used. This can be accomplished by the use of a compression material, such as that available under the trademark POWERNET, that when worn in the manner proposed by the present invention, provides uniform compression through the mid-abdomen.

Thus, an object of the invention is to disclose a method for using a compression element under the supervision of a medical practitioner to provide measurable reductions in appetite by the wearer while facilitating weight loss without the need for surgery or medication.

A further object of the present invention is to provide a medically proven means of promoting weight loss through the application of a safe, modest, and comfortable pressure load on the mid-abdomen during normal, everyday activities.

Another object of the invention is to teach the use of a compression garment to provide compression directly to the mid-abdomen with minimum discomfort to the surrounding areas allowing wearer's to keep the garment on for prolonged periods of time.

Still another object of the present invention is to teach the use of a compression garment that is comprised of breathable material and allows for stretching to provide a comfortable garment that may be worn under one's clothing.

Another object of the invention is to teach the use of a compression garment with a plurality of adjustment members for adjusting the level of compression without changing the garment.

The present invention provides a noninvasive method and apparatus for facilitating weight loss that obviates the need for surgery or medication in many individuals struggling to overcome issues associated with excessive weight. The invention is comfortable enough to be used every day without causing undue agitation to the wearer and provides measurable reductions in appetite and increased weight loss for users. Numerous other features, objects and advantages of the invention will become apparent from the following description when read in conjunction with the accompanying drawings.

### DESCRIPTION OF THE DRAWINGS

### Figures

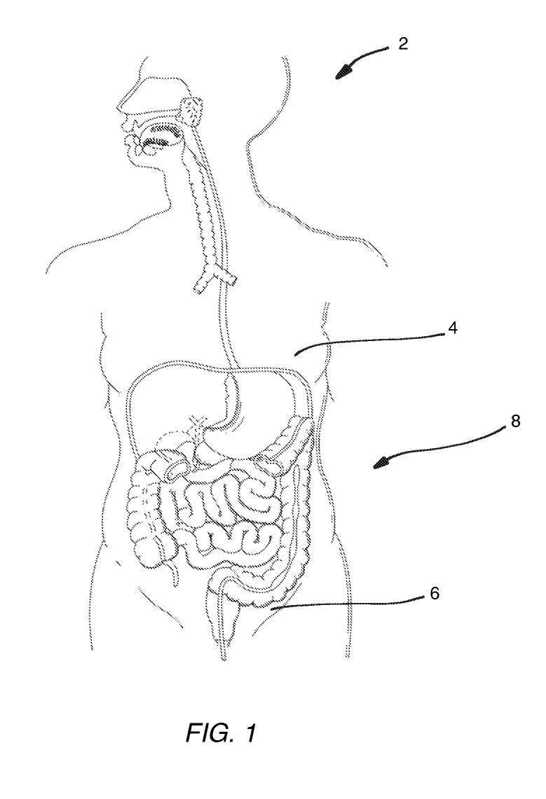

FIG. 1 is a partial cross-sectional view of the human body illustrating portion of the gastrointestinal tract;

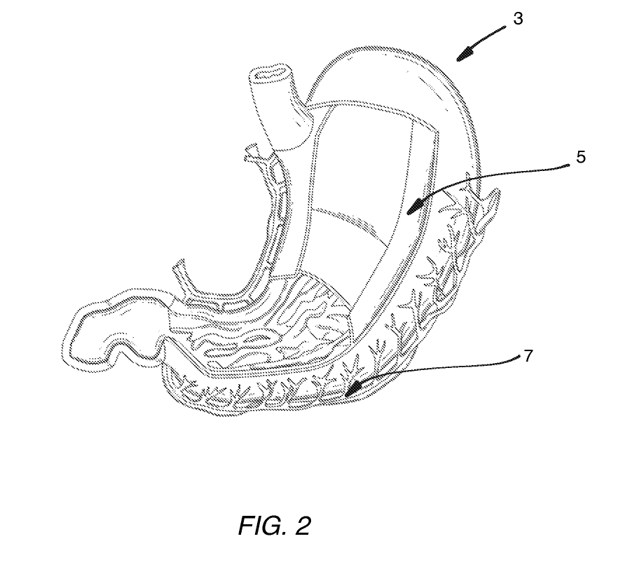

FIG. 2 is a detailed cross sectional profile view of a human stomach illustrating the layers of tissue enveloping it with some of the surrounding vasculature;

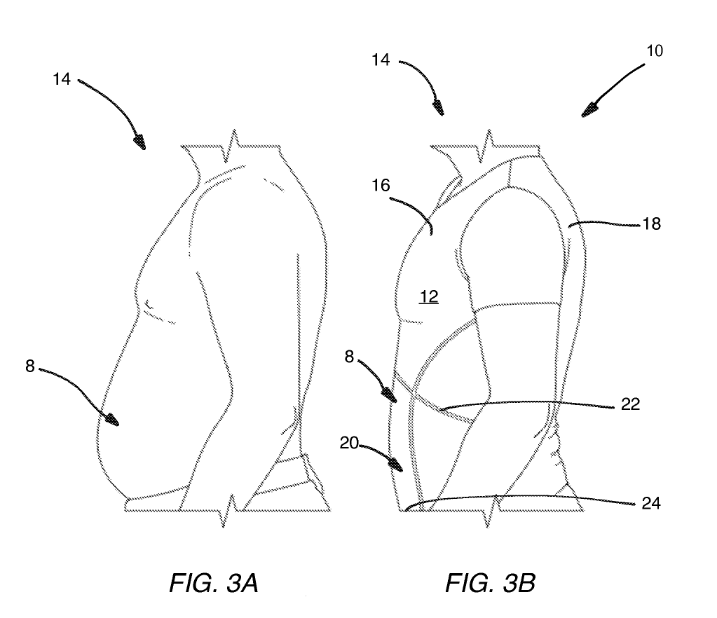

FIGS. 3A & 3B illustrate the compression effect the apparatus of the present invention has on the wearers stomach;

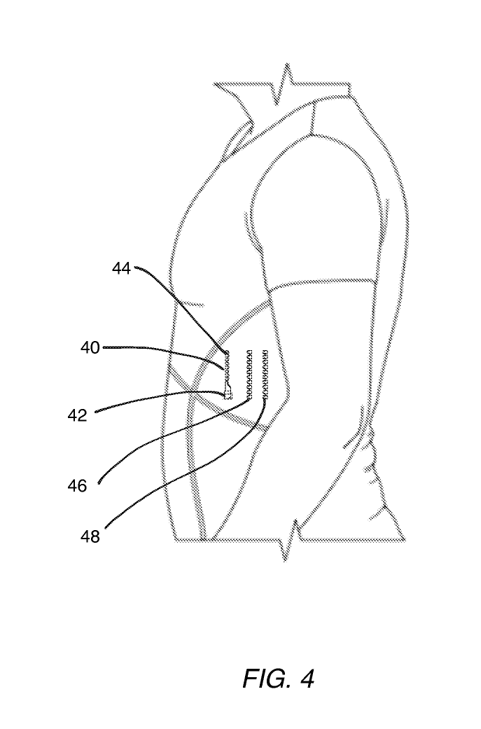

FIG. 4 is a profile view of an alternative embodiment employing zippers for adjustment of the apparatus;

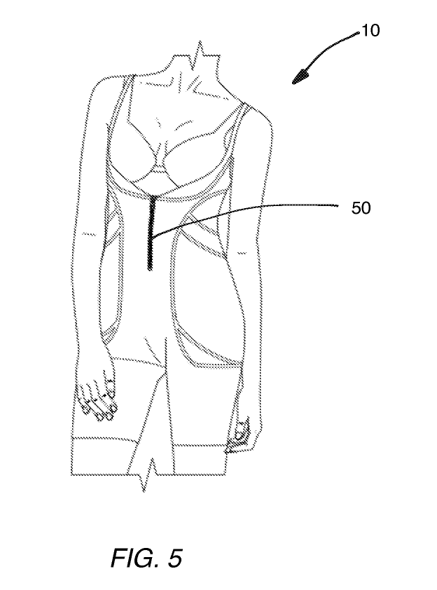

FIG. 5 is a perspective view of an alternative embodiment of the apparatus exemplified as a unitard;

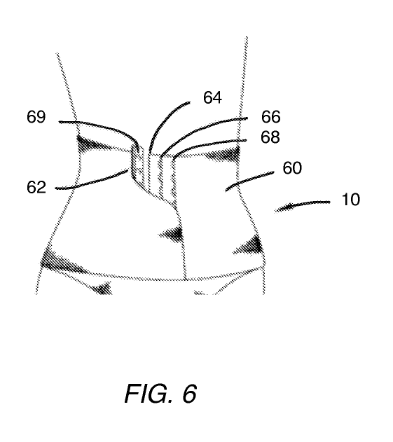

FIG. 6 is a rear view of an alternative embodiment employing hooks for adjustment;

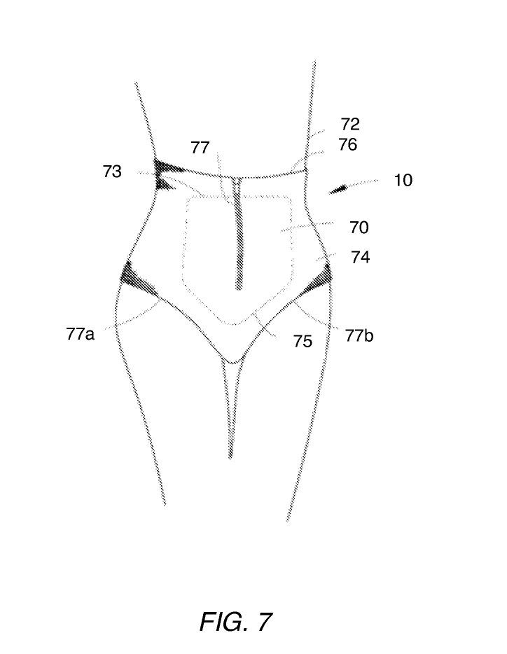

FIG. 7 is a frontal view of an alternative embodiment of the present invention employing a frontal opening;

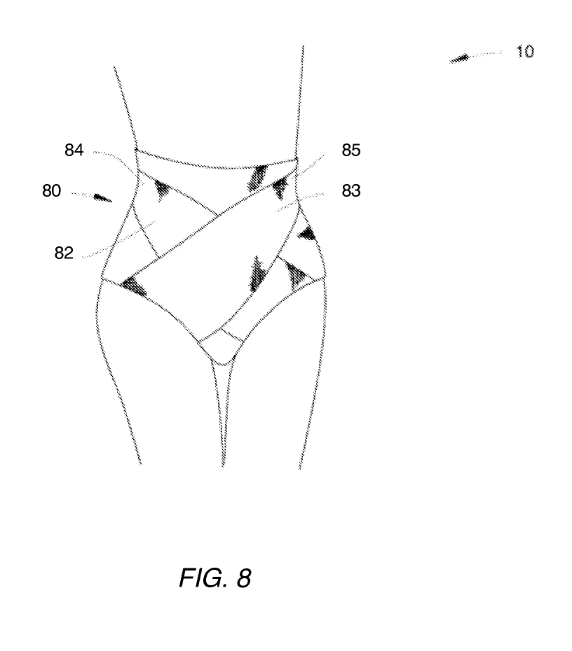

FIG. 8 depicts an alternate embodiment having releasable fasteners;

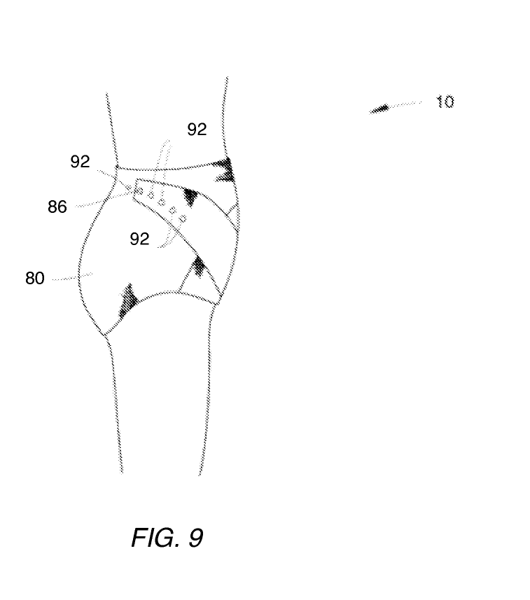

FIG. 9 is a side view of the apparatus in FIG. 8; and

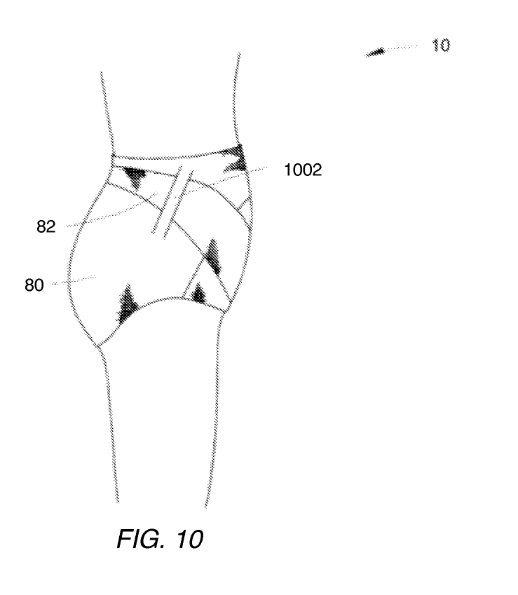

FIG. 10 depicts an alternate embodiment of the apparatus, comprising positioning members.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein.

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Illustrative embodiments of the invention are described below. The showings are for purposes of illustrating preferred embodiments and not for purposes of limiting the same. The following explanation provides specific details for a thorough understanding of an enabling description for these embodiments. One skilled in the art will understand that the invention may be practiced without such details.

The following method facilitates weight loss in human beings using specifically directed topical pressure. In one embodiment, the method begins with topical pressure applied via a compression apparatus to a plurality of predetermined locations on a patient following a weight loss regiment. The locations of the body 2 for receiving the directed pressure are depicted in FIGS. 1 and 2 as defined generally by the area of the patient's body between the mid-sternum 4 and the pubic bone 6 heretofore referred to as the mid-abdomen 8. FIG. 2 is a detailed cross sectional profile view of a human stomach 3 illustrating the layers of tissue 5 enveloping it with some of the surrounding vasculature 7.

Even though FIGS. 1 and 2 only indicate locations on the front side of the body 2, those skilled in the art will realize these locations are mirrored on the back side of the body as well. Consequently, when the locations for targeted pressure are mentioned, it is generally understood that the named locations can also include that same location on the opposite side of the human body 2.

Typically, pressure is applied for at least 6 hours per day while the patient is awake using a compression apparatus 10. In the preferred embodiment, the compression apparatus 10 is a compression garment (various exemplary embodiments depicted in FIGS. 3-10). If worn regularly, the apparatus 10 will facilitate weight loss by mimicking the feeling one gets when they are full thus reducing the wearer's appetite.

The amount of time for the pressure treatment may vary. It is understood that a physician overseeing the patient's care may vary the length of time and the amount of time the pressure is applied. It is further understood that these normal variations are foreseeable by those of ordinary skill in the art and are covered under the present invention. At the conclusion of the treatment, the patient is instructed to discontinue use of the compression apparatus 10.

The efficacy of the present invention has been shown using a series of single-blind experiments under the supervision of licensed physicians. Using twenty-four patients over the course of eighty-four days, patients were monitored to evaluate the change in percentage of weight loss between those who were and were not using the compression apparatus 10 of the present invention. The study revealed those using the compression apparatus 10 experienced substantially higher amounts of weight lost during the eighty-four day period. The average recorded increase in patient weight loss for those using the compression apparatus 10 was approximately twenty percent.

FIG. 3A illustrates the stomach of a person not wearing the apparatus 10. FIG. 3B is an illustration of an abdominal weight loss apparatus 10, in accordance with a first exemplary embodiment of the present disclosure. In accordance with the aspects of the present invention, FIG. 3B depicts the apparatus 10 in the form of a garment 12 as worn by a person 14 having a front side 16 and a back side 18. The apparatus 10 is adapted to fit securely and apply pressure directed to the person's mid-abdomen 8.

The abdominal weight loss apparatus 10 includes a flexible abdominal covering structure 20 sized to cover and fully surround a mid-abdomen 8. The abdominal covering structure 20 has a substantially tubular body ending in an upper edge 22 and a lower edge 24. The abdominal covering structure 20 exerts directed topical pressure proximate to the mid-abdomen 8. The mid-abdomen 8 may be larger or smaller in different individuals. Accordingly, the abdominal covering structure 20 may also be a variety of different lengths, widths, or thicknesses to accommodate people of different sizes.

As illustrated in FIGS. 3-10, the garment may have a number of different shapes designed to fit both male and female patients. All embodiments may be successfully used with the method disclosed herein. It is also understood that various additional shapes and types of garments would be acceptable; provided that they exert suitable pressure to surface of the patient's skin in the region defined as the mid-abdomen.

In one embodiment, the garment is constructed of an elastomeric material. When selecting compression materials for use with the disclosed method, the strength, lightness, and elasticity of the material are all important. The material must be comfortable enough to be worn for a prolonged period of time. Thus, it should be porous to facilitate heat and air exchange. The garment must comprise a material capable of exerting the desired amount of pressure on the mid-abdomen without constricting wearer mobility so much that it substantially interferes with any wake time activities.

In one embodiment of the present invention, the elastomeric material may be of the type designed to provide a form fitting shape, such as those available under the trademark SPANDEX. Although this material may be used in conjunction with other materials to shape a variety of different garments, all such garments of the present invention are to comprise an area of high elasticity providing maximum compression around an area defined by the mid-abdomen of the wearer.

In a further embodiment, the garment formed from a straight compression material such as that sold under the trademark SPANX. The compression material may include a form fit that targets the maximum amount of compression to the mid-abdomen 8 of the wearer.

FIG. 4 illustrates another exemplary embodiment of the apparatus 10. By way of example, and not limitation, the apparatus 10 depicted includes a zipper handle 42 which is secured to vertical zipper line 44. When left unattached, the zipper attachment 40 provides minimum compression. When zipper handle 42 is attached to second vertical zipper line 44, the spatial distance between zipper line 46 and 48 is removed providing increased compression. When zipper handle 42 is attached to a third vertical zipper line 48, the spatial distance between zipper line 44 and 48 is removed providing maximum compression. This compression adjustment further allows the individual more comfort during the positioning process. The garment may consist of other similar devices for adjusting the size including snaps, hook-and-eye closures, hook-and-loop type fastening material, or the like.

In another embodiment, the compression apparatus 10 may also be made adjustable to provide a range of compression to the mid-abdomen area 8 with minimum discomfort to the wearer. Now referring to FIG. 5, in order to increase, reduce or maintain compression on the abdomen throughout the weight loss process, the compression apparatus 10 may contains a vertical zipper attachment 50 placed along the front of the apparatus for tightening or loosening the apparatus 10 around the desired body area. As the abdomen returns to the desired size, the zippers can be adjusted by zipping the zipper to a certain degree to maintain pressure on the desired part of the body within the apparatus. The zipper or zipper attachments may be zipped to any particular point along the zipper area and held in that position by pressing the zipper flap (not shown) down into a secure position. The apparatus may be either tightened or loosened in the mid-abdomen by again zipping up or down the zipper to a position that is desired by the person wearing the apparatus 10 or the medical practitioner overseeing their care.

In another embodiment of the present invention depicted in FIG. 6, the compression apparatus 10 may be comprised of a singular wrap of compression material having a first end 62 and second end 64 connected by a plurality of fastening members 66 permitting the wearer to easily wear and remove the compression apparatus 10 from their mid-abdomen 8. FIG. 6 depicts an exemplary embodiment of the apparatus 10 in an open position illustrating a line of eyes 68 placed adjacent to the end 64 and a second line of eyes 68 placed adjacent to the first set allowing manual adjustment of fastening hooks 69 in relation to the amount of compression desired or required.

FIG. 7 depicts yet another embodiment of the present invention with apparatus 10 as worn by the wearer 72 having a front side 74 and a back side, not shown. The apparatus 10 is adapted to fit the wearer's body having a tubular shape with a torso opening 76 and first and second leg openings 77a, 77b. This embodiment is illustrated as a garment constructed of elastic material, providing a form fitting shape. The front side 76 includes an area 70 of high elasticity designed to provide the maximum compression to the mid-abdomen 8. The area 70 is of an elasticity higher than the remaining garment providing an increased area of compression between upper end 73 and lower end 75. In this manner, the bulk of the compression remains directed to the central area 70 provides uniform compression in multiple directions yet tends to while pushing the mid-abdomen 8. Moreover, apparatus may comprise various means for frontal garment attachment such as a zipper 77, buttons, hook-and-loop fastening means and the like.

FIG. 8 illustrates another embodiment of a compression apparatus 10 exemplified as a garment comprising a support structure sized to conform to the anatomical shape of the wearer and directing pressure to the wearers mid-abdomen. In this embodiment, the garment 80 includes a plurality of elastomeric strips 82 and 83 that help adjust the amount of direct compression produced by the garment 80. The strips 82 and 83 may cross each other, and each has a distal free end 84 and 85. The distal free ends 84 and 85 each include a releasable fastener first portion 86 (See FIG. 9).

As shown in FIG. 9, the garment 80, itself, includes releasable fastener second portions 92 located along the surface of the garment 80. The distal free ends 84 and 85 may be releasably secured to several locations along the surface of the garment 80. Accordingly, the wearer may customize the amount of topical compression produced by the garment 80. This allows the garment 80 to fit correctly even as the wearer's proportions change.

With reference to the embodiment illustrated in FIG. 10, the garment 80 may further include positioning members 1002 that accommodate the elastic strips 82 and 83, holding the strips against the exterior surface of the garment 80. The positioning members 1002 may be formed integral with the garment 80 or may be discrete loops or flaps attached thereto.

The support structure is worn by a person while undergoing a weight loss program and the compression means provides topical pressure to the mid-abdominal region mimicking the sensation of being full.

Although the invention has been described with reference to specific embodiments, it will be understood by those skilled in the art that various changes may be made without departing from the scope of the invention. Accordingly, the disclosure of embodiments is intended to be illustrative of the scope of the invention and is not intended to be limiting. It is intended that the scope of the invention shall be limited only to the extent required by the appended claims. To one of ordinary skill in the art, it will be readily apparent that the devices and method discussed herein may be implemented in a variety of embodiments, and that the foregoing discussion of certain of these embodiments does not necessarily represent a complete description of all possible embodiments. Rather, the detailed description of the drawings, and the drawings themselves, disclose at least one preferred embodiment, and may disclose alternative embodiments.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents

### CONCLUSIONS, RAMIFICATIONS, AND SCOPE

While a particular form of the invention has been illustrated and described, it will be apparent that various modifications can be made without departing from the spirit and scope of the invention. For example, the compression apparatus is illustrated as being comprised of material available under the trademark SPANDEX in some embodiments even though the inventor contemplates the possibility that it may comprised of material available under the trademark POWERNET all the while comprising the properties of the invention. Accordingly, is not intended that the invention be limited, except as by the appended claims.

The teachings provided herein can be applied to other systems, not necessarily the method and apparatus described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Particular terminology used when describing certain features or aspects of the invention should not be taken to imply that the terminology is being refined herein to be restricted to any specific characteristics, features, or aspects of the noninvasive method and apparatus for facilitating weight loss with which that terminology is associated. In general, the terms used in the following claims should not be constructed to limit the method and apparatus to the specific embodiments disclosed in the specification, unless the above description section explicitly define such terms. Accordingly, the actual scope encompasses not only the disclosed embodiments, but also all equivalent ways of practicing or implementing the disclosed method and apparatus. The above description of embodiments of the method and apparatus is not intended to be exhaustive or limited to the precise form disclosed above or to a particular field of usage. While specific embodiments of, and examples for, the noninvasive method and apparatus for facilitating weight loss are described above for illustrative purposes, various equivalent modifications are possible which those skilled in the relevant art will recognize.

While certain aspects of the noninvasive method and apparatus for facilitating weight loss are presented below in particular claim forms, the inventor contemplates the various aspects of the method and apparatus in any number of claim forms. Accordingly, the inventor reserves the right to add additional claims after filing the application to pursue such additional claim forms for other aspects of the noninvasive method and apparatus for facilitating weight loss.

What is claimed is:

1. A method of facilitating weight loss in a person, said method comprising the steps of:
applying topical pressure through a non-invasive pressure apparatus to a plurality of predetermined locations on said person's body, including areas comprising the mid-abdomen; and
removing said pressure apparatus after a set time.

2. The method according to claim 1, wherein a set time is at least 6 hours.

3. The method of claim 1 wherein said non-invasive pressure apparatus is a compression garment.

4. The method of claim 3 wherein said compression garment comprises a flexible abdominal covering structure sized to cover and fully surround a human mid-abdomen, said abdominal covering structure having a substantially tubular body ending in an upper edge and a lower edge, wherein said abdominal covering structure exerts topical pressure proximate to said mid-abdomen; and where said covering is comprised of an elastomeric material.

5. The garment of claim 4 wherein said compression garment is a one piece suit having upper body and lower body portions and arms and leg portions.

6. The method of claim 1 further comprising the steps of said person performing routine physical activities while wearing said apparatus wherein said activities include movement of at least some portion of the body having said apparatus thereon, said apparatus applying pressure to the mid-abdomen of said person during said physical activities to mimic the sensation of being full, and wearing the apparatus continuously for an extended period of time.

7. The method of claim 6 wherein said apparatus is worn as part of a weight loss program and including the step of changing said apparatus to a further resistance apparatus having different resistance characteristics during said weight loss program.

8. The method of claim 3 wherein said compression garment comprises a support structure sized to conform to the anatomical shape of said person between the mid-sternum and the pubic bone; compression means positioned along a frontal portion of said structure, said compression means being defined as a plurality of elastomeric strips positioned so as to cause a direct compression to the mid-abdominal region of said person when said garment is worn, said elastomeric strips are elongated and placed in a manner so as to provide highest compression at said mid-abdomen;
at least one releasable fastener first portion disposed on a distal free end of each said elastomeric strip;
a plurality of releasable fastener second portions, said releasable fastener second portions constructed and arranged to selectively engage said fastener first portions, said compression garment further including a means for adjusting said strips to provide a variance in the amount of said direct compression provided by the garment; and a plurality of positioning members sized to accommodate said elastomeric strips;
wherein said support structure is worn by said person while undergoing a weight loss program whereby said compression means provides topical pressure to the mid-abdominal region to mimic the sensation of being full and whereby said releasable fastener second portions provide preferentially defined locations for attachment of said releasable fastener first portions, thereby allowing adjustment of said direct compression, and whereby when said elastomeric strips pass through said positioning members, said positioning members keep said elastomeric strips in touching contact with the exterior surface of said compression garment.

9. A method of facilitating weight loss in a person, said method comprising the steps of:
providing an elastomeric member,
using said member to apply sufficient topical pressure to the skin of said person to mimic the sensation of being full without the use of surgery or medication.

10. The method of claim 9 wherein said pressure is applied to the mid-abdomen of said person

11. The method of claim 9 wherein said member is comprises a plurality of adjustment members.

12. The method of claim 9 wherein said elastomeric member is a compression garment comprising a flexible abdominal covering structure sized to cover and fully surround a human mid-abdomen, said abdominal covering structure having a substantially tubular body ending in an upper edge and a lower edge, wherein said abdominal covering structure exerts topical pressure proximate to said mid-abdomen; and where said covering is comprised of an elastomeric material.

13. The method of claim 9 further comprising the steps of said person performing routine physical activities while wearing said elastomeric member wherein said activities include movement of at least some portion of the body having said member thereon, said member applying pressure to the mid-abdomen of said person during said physical activities to mimic the sensation of being full, and wearing the member continuously for an extended period of time.

14. The method of claim 9 wherein said elastomeric member is a compression garment comprising a support structure sized to conform to the anatomical shape of said person between the mid-sternum and the pubic bone; compression means positioned along a frontal portion of said structure, said compression means being defined as a plurality of elastomeric strips positioned so as to cause a direct compression to the mid-abdominal region of said person when said garment is worn, said elastomeric strips are elongated and placed in a manner so as to provide highest compression at said mid-abdomen;
at least one releasable fastener first portion disposed on a distal free end of each said elastomeric strip;
a plurality of releasable fastener second portions, said releasable fastener second portions constructed and arranged to selectively engage said fastener first portions, said compression garment further including a means for adjusting said strips to provide a variance in the amount of said direct compression provided by the garment; and a plurality of positioning members sized to accommodate said elastomeric strips;
wherein said support structure is worn by said person while undergoing a weight loss program whereby said compression means provides topical pressure to the mid-abdominal region to mimic the sensation of being full and whereby said releasable fastener second portions provide preferentially defined locations for attachment of said releasable fastener first portions, thereby allowing adjustment of said direct compression, and whereby when said elastomeric strips pass through said positioning members, said positioning members keep said elastomeric strips in touching contact with the exterior surface of said compression garment.
